(19) United States
(12) Patent Application Publication
(10) Pub. No.: US 2017/0072158 A1
(43) Pub. Date: Mar. 16, 2017

(54) CLOSED LOOP AIR-OXYGEN BLENDER WITH HIGH AND LOW PRESSURE AIR INLET

(71) Applicant: 12th Man Technologies, Inc., Garden Grove, CA (US)

(72) Inventors: Steve Han, Huntington Beach, CA (US); Alex Stenzler, Long Beach, CA (US)

(21) Appl. No.: 15/267,266

(22) Filed: Sep. 16, 2016

Related U.S. Application Data
(60) Provisional application No. 62/219,209, filed on Sep. 16, 2015.

(51) Int. Cl. A61M 16/12; A61M 16/08; A61M 16/20

(52) U.S. Cl. CPC A61M 16/125; A61M 16/203; A61M 16/208; A61M 16/0875; A61M 2016/003

(57) ABSTRACT

An electronic gas blender system includes a pressurized oxygen supply connected in-line to a first proportional valve, a first check valve and a first flow sensor by a first circuit of gas lines, a pressurized air supply connected in-line to a second proportional valve, a second check valve and a second flow sensor by a second circuit of gas lines, and a port disposed between the second check valve and the second flow sensor on the second circuit of gas lines for providing airflow access to a low pressure source. The port includes a plug configured to open and close the airflow access, and a control module is electrically coupled to the first proportional valve, the first flow sensor, the second proportional valve, and the second flow sensor. The electronic gas blender system can be used to blend two high pressure gas sources under dynamic control or one high pressure and one zero or low pressure gas flow source through a slave/master relationship.

### CROSS-REFERENCE TO RELATED APPLICATIONS

This application claims priority to U.S. provisional application No. 62/219,209 filed on Sep. 16, 2015 incorporated herein by reference in its entirety.

### BACKGROUND OF THE INVENTION

Gas blenders used for mixing medical gases such as room air and oxygen typically use two high pressure gas sources and use proportional controllers to determine the final concentration of the delivered gas. These can be mechanical blenders such as that described in DeVries (U.S. Pat. No. 5,014,694), or the blender can be electronically controlled, such as the microprocessor controlled blender disclosed in Lampotang et al. (U.S. Pat. No. 5,887,611) or the electronic blender disclosed in Volgyesi (U.S. Pat. No. 6,857,443). Typically, the mechanical blenders use either balancing blocks with high pressure input (for example ˜40-60 psig), or a high pressure venture design that can deliver a constant proportion of air and oxygen (i.e., percent oxygen) based on the relative outlet of each. The gas lines used to support this high pressure design are usually small in diameter for providing a high enough resistance to enable control of pressure for delivering a precise flow. The same is true for the electronic blenders that depend on the high pressure and a variable orifice to set the proportion of each gas. However, if oxygen is desired to be mixed into a stream of air for spontaneously breathing subjects, the small diameter delivery lines or the proportional valves make it impossible for the patients to breathe through.

Conventional electronically controlled gas blenders typically have a microprocessor that, based upon knowing the desired flow and desired concentration, can determine the correct proportion of each gas. Accordingly, these systems will open a proportional valve for each gas under microprocessor control, allowing the correct proportion of each gas to flow. These can be control valves for an open loop or closed loop system that follow dynamic input waveforms from a microprocessor. However, these conventional systems do not allow the dynamic blending of gas during spontaneous breathing since these are high resistance systems where all of the flow patterns for each gas channel are controlled by the microprocessor. There are also spontaneous breathing gas mixers whereby the oxygen can be injected into an airstream proportional to the spontaneous breathing flow, but only if the oxygen injection is controlled by a closed loop feedback loop based on a low resistance spontaneous breathing path. Unfortunately, there are no blenders that can easily perform both a high pressure blending mode and spontaneous breathing mode for dynamic mixing.

What is needed in the art is a blender that enables use of high pressure gas lines for a high pressure blending mode that can be easily converted to a low resistance spontaneous breathing mode for dynamic mixing.

### SUMMARY OF THE INVENTION

In one embodiment, a gas blender system includes a pressurized oxygen supply connected in-line to a first proportional valve, a first check valve and a first flow sensor by a first circuit of gas lines, a pressurized air supply connected in-line to a second proportional valve, a second check valve and a second flow sensor by a second circuit of gas lines, a port disposed between the second check valve and the second flow sensor on the second circuit of gas lines for providing airflow access to a low pressure source, where the port comprises a plug configured to open and close the airflow access, and a control module electrically coupled to the first proportional valve, the first flow sensor, the second proportional valve, and the second flow sensor. In one embodiment, the low pressure source is ambient room air. In one embodiment, the control module is electrically coupled to the plug and is configured to open the airflow access during a spontaneous breathing state and close the airflow access during a high pressure blending state. In one embodiment, during a spontaneous breathing state, the control module is configured to adjust the first proportional valve based on an airflow from a low pressure source measured at the second flow sensor. In one embodiment, the control module is a microprocessor. In one embodiment, the control module is an analog circuit. In one embodiment, the port is at least 10 millimeters in diameter at its narrowest point. In one embodiment, the first circuit of gas lines is less than 3 millimeters in diameter at its narrowest point. In one embodiment, the low pressure source is pressurized to less than 5 psi. In one embodiment, the low pressure source is pressurized to less than 4 psi. In one embodiment, the low pressure source is pressurized to less than 3 psi. In one embodiment, the low pressure source is pressurized to less than 2 psi. In one embodiment, the low pressure source is pressurized to less than 1 psi. In one embodiment, the low pressure source is unpressurized air.

In one embodiment, a method for dynamic mixing of gases includes the steps of providing a pressurized oxygen supply connected to a first circuit of gas lines, and a pressurized air supply connected to a second circuit of gas lines, wherein the first and second circuit of gas lines converge on a user inhalation gas line; supplying a mix of the pressurized oxygen and the pressurized air in the user inhalation gas line during a pressurized blending mode; and supplying a mix of the pressurized oxygen and ambient air in the user inhalation gas line during a spontaneous breathing mode. In one embodiment, the method includes the step of preventing user inhalation gas line access to the pressurized air supply during the spontaneous breathing mode. In one embodiment, the method includes the step of closing an access port to ambient air or a low pressure air source during the pressurized blending mode. In one embodiment, the method includes the step of switching between the pressurized blending mode and the spontaneous breathing mode based on an electrical signal received from a flow sensor. In one embodiment, a source of the low pressure air is pressurized to less than 5 psi. In one embodiment, the low pressure source is pressurized to less than 4 psi. In one embodiment, the low pressure source is pressurized to less than 3 psi. In one embodiment, a source of the low pressure air is pressurized to less than 2 psi. In one embodiment, a source of the low pressure air is pressurized to less than 1 psi. In one embodiment, a source of the low pressure air is unpressurized.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a closed loop system that enables both a high pressure gas blending mode and a low resistance spontaneous breathing mode for dynamic mixing according to one embodiment.

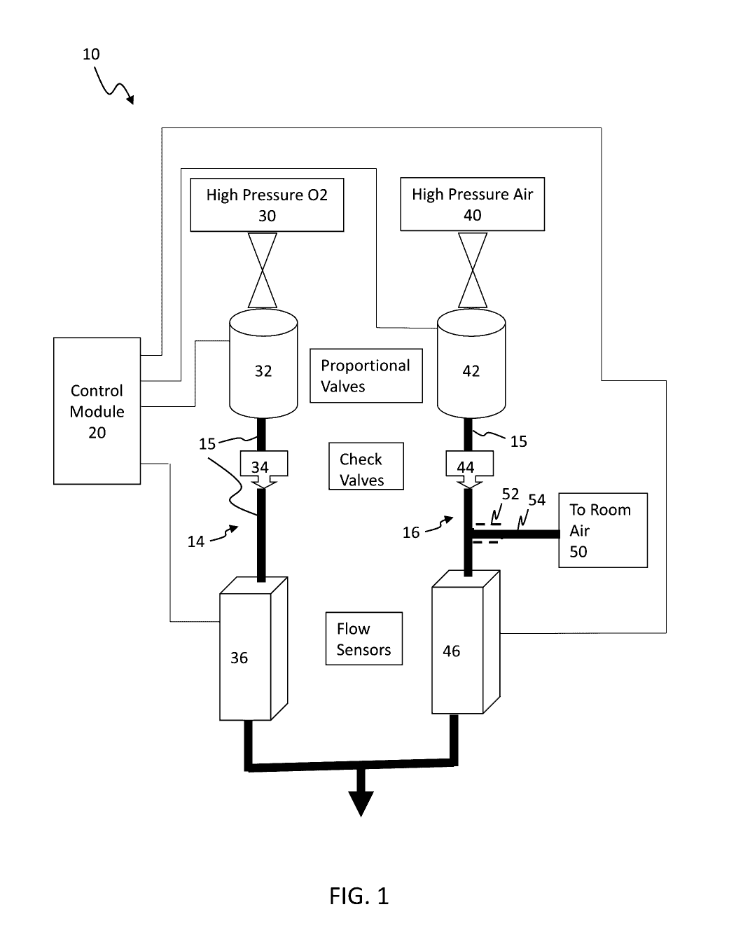

FIG. 2 is a flow chart illustrating a pressurized blending mode and a spontaneous breathing mode according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in systems for both high pressure gas blending and low resistance spontaneous breathing. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Low pressure” includes gas or air sources at pressure differentials of 0-5 psi.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein are systems and methods for permitting both high pressure gas blending and low resistance dynamic mixing for spontaneous breathing.

In one embodiment, the gas blender system 10 includes a high pressure oxygen supply 30 connected in-line to a first proportional valve 32, a first check valve 34 and a first flow sensor 36 connected by high pressure gas lines 15. The first proportional valve 32 and the first flow sensor 36 are electrically coupled to a control module 20. In one embodiment, the control module is a microprocessor. In another embodiment, the control module is analog circuitry. In one embodiment, the high pressure gas lines 15 have a diameter of between 1 and 5 mm at its narrowest point. The gas blender system 10 also includes a high pressure air supply 40 connected in-line to a second proportional valve 42, a second check valve 44 and a second flow sensor 46. The second proportional valve 42 and the second flow sensor 46 are electrically coupled to the control module 20.

In one embodiment, when the gas blender system 10 is operating in the high pressure supply mode, the control module 10 sends a signal to the gas mixing circuitry and related electromechanical components connected to the proportional valves 32, 42 for controlling the total gas flow and the proportion of the gas that is oxygen. The gas mixing circuitry and electromechanical components will open each of the proportional valves 32, 42 to the opening required to deliver the set concentration and total flow. In certain embodiments, the circuit controlling each gas flow includes a proportional valve 32, 42 (for example a proportional solenoid valve or a voltage sensitive orifice), a one way check valve on the inlet 34, 44 for preventing the backflow of gas, and a flow sensor 36, 46 on the outlet of each circuit. The flow sensors 36, 46 confirm that the flow leaving the valve is exactly was it is supposed to be. If the measured flow is off from the target flow, the closed loop circuit for the valve control will adjust the one or both of the proportional valve settings so that the valve output is corrected to the expected flow. This correction is performed very quickly and is typically within 1 millisecond per cycle. This can be set to fixed flow and concentration or it can provide mixed gas at a varying flow under microprocessor control.

In one embodiment, when the gas blender system 10 circuit is being used as a low pressure circuit for blending oxygen into a spontaneously breathing patient's air stream, a plug 52 is opened into the blender between the outlet of the proportional valve 42 and the flow sensor 46. The plug 52 provides gas access to a port 54 which allows air from a low pressure source, such as ambient air or room air 50 to be drawn into the blender 10, measured by the second flow sensor 46 and oxygen injected into the circuit to keep the concentration fixed. A gas such as air for the low pressure circuit can be provided by, for example, ambient room air, a container holding air at a low pressure, or a ventilator. In one embodiment, low pressure is a pressure differential of less than 5 psi. In other embodiments, low pressure is a pressure differential of less than 4, 3, 2 or 1 psi. In one embodiment, the low pressure source is an unpressurized source, such as ambient room air. In certain embodiments, the port 54 has a minimum diameter of 10 or 15 mm at its narrowest point. In certain embodiments, the plug is controlled by an electromechanical component that is electrically coupled to the control module. The injecting, mixing and closed loop control allows the oxygen to be injected into the circuit directly proportional to the room air flow. In this configuration, the second flow sensor 46 is the master signal for the oxygen injector's first proportional valve 32, which functions as a slave to the air flow signal while the oxygen flow signal measured at the first flow sensor 36 is used for closed loop feedback on oxygen injection. A minimal amount of air flow must be measured to control oxygen injection. In one embodiment, a minimum of 5% of the gas must be from the room pressure (low pressure) air source must be measured to activate and control oxygen injection. In one embodiment, the gas blender system 10 includes a high pressure closed loop oxygen supply circuit and a high or low pressure closed loop air supply circuit that enables dynamic blending under microprocessor 20 control from 21% to 100% oxygen when using both supply gases as high pressure sources or from 21% to 95% oxygen when using only oxygen as the high pressure source for spontaneously breathing patients.

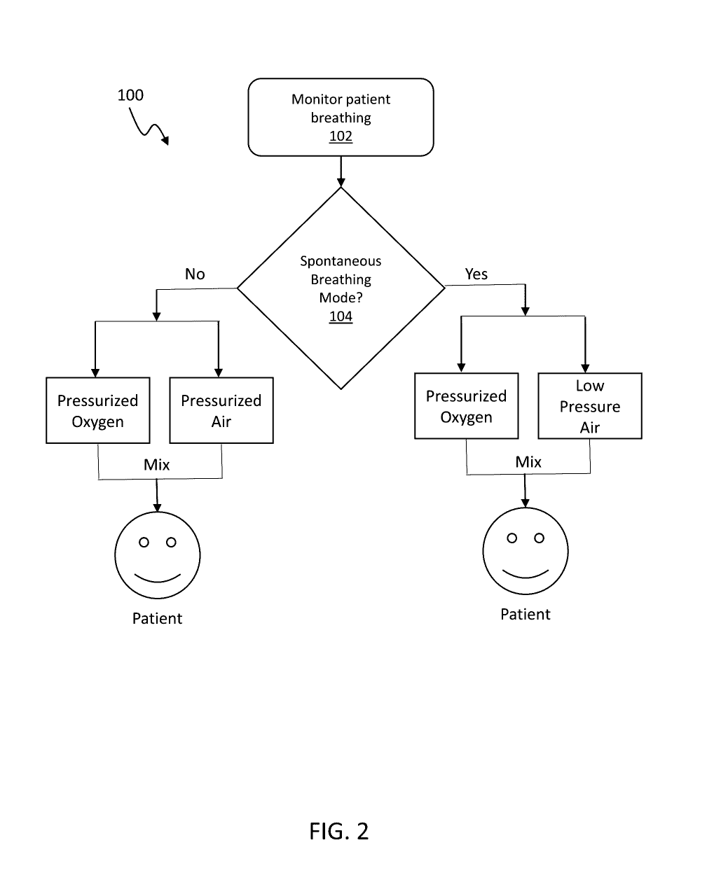

A method for dynamic mixing of gases 100 according to one embodiment is provided in the flow chart of FIG. 2. Patient breathing can be monitored 102 by observation of a medical professional, or automatically by the system, through for example a flow sensor that can sense an inlet flow form a low pressure air source (e.g. room air), or a flow sensor near the patient's mouth. If it is determined 104 that the patient can breathe on their own, the spontaneous breathing mode is activated. If it is determined 104 that the patient cannot breathe on their own, the pressurized blending mode is activated. In one embodiment, a pressurized oxygen supply is connected to a first circuit of gas lines, and a pressurized air supply is connected to a second circuit of gas lines. The first and second circuit of gas lines converge on a user inhalation gas line, supplying a mix of the pressurized oxygen and the pressurized air in the user inhalation gas line during a pressurized blending mode, and supplying a mix of the pressurized oxygen and ambient air in the user inhalation gas line during a spontaneous breathing mode. In one embodiment, the user inhalation gas line is prevented from accessing the pressurized air supply during the spontaneous breathing mode. In one embodiment, an access port to ambient air or a low pressure air source is closed during the pressurized blending mode.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A gas blender system comprising:
a pressurized oxygen supply connected in-line to a first proportional valve, a first check valve and a first flow sensor by a first circuit of gas lines,
a pressurized air supply connected in-line to a second proportional valve, a second check valve and a second flow sensor by a second circuit of gas lines,
a port disposed between the second check valve and the second flow sensor on the second circuit of gas lines for providing airflow access to a low pressure source, wherein the port comprises a plug configured to open and close the airflow access, and
a control module electrically coupled to the first proportional valve, the first flow sensor, the second proportional valve, and the second flow sensor.

2. The gas blender system of claim 1, wherein the low pressure source is ambient room air.

3. The gas blender system of claim 1, wherein the control module is electrically coupled to the plug and is configured to open the airflow access during a spontaneous breathing state and close the airflow access during a high pressure blending state.

4. The gas blender system of claim 1, wherein during a spontaneous breathing state, the control module is configured to adjust the first proportional valve based on an airflow from a low pressure source measured at the second flow sensor.

5. The gas blender system of claim 1, wherein the control module is a microprocessor.

6. The gas blender system of claim 1, wherein the control module is an analog circuit.

7. The gas blender system of claim 1, wherein the port is at least 10 millimeters in diameter at its narrowest point.

8. The gas blender system of claim 7, wherein the first circuit of gas lines is less than 3 millimeters in diameter at its narrowest point.

9. The gas blender system of claim 1, wherein the low pressure source is pressurized to less than 5 psi.

10. The gas blender system of claim 1, wherein the low pressure source is pressurized to less than 2 psi.

11. The gas blender system of claim 1, wherein the low pressure source is pressurized to less than 1 psi.

12. The gas blender system of claim 1, wherein the low pressure source is unpressurized air.

13. A method for dynamic mixing of gases comprising:
providing a pressurized oxygen supply connected to a first circuit of gas lines, and a pressurized air supply connected to a second circuit of gas lines, wherein the first and second circuit of gas lines converge on a user inhalation gas line, and supplying at least one of:
a mix of the pressurized oxygen and the pressurized air in the user inhalation gas line during a pressurized blending mode; and
a mix of the pressurized oxygen and low pressure air in the user inhalation gas line during a spontaneous breathing mode.

14. The method of claim 9 further comprising:
preventing user inhalation gas line access to the pressurized air supply during the spontaneous breathing mode.

15. The method of claim 9 further comprising:
closing an access port to the low pressure air during the pressurized blending mode.

16. The method of claim 9, further comprising:
switching between the pressurized blending mode and the spontaneous breathing mode based on an electrical signal received from a flow sensor.

17. The gas blender system of claim 1, wherein a source of the low pressure air is pressurized to less than 5 psi.

18. The gas blender system of claim 1, wherein a source of the low pressure air is pressurized to less than 2 psi.

19. The gas blender system of claim 1, wherein a source of the low pressure air is pressurized to less than 1 psi.

20. The gas blender system of claim 1, wherein a source of the low pressure air is unpressurized.
